(19) United States
(12) Patent Application Publication
(10) Pub. No.: US 2026/0351279 A1
(43) Pub. Date: Nov. 5, 2026

(54) ELECTRONIC DEVICE AND OPERATION METHOD FOR IDENTIFYING STATE OF A USER

(71) Applicant: SAMSUNG ELECTRONICS CO., LTD., Suwon-si (KR)

(72) Inventors: Byeol PARK, Suwon-si (KR); Daewon YOON, Suwon-si (KR); Jinhyun KIM, Suwon-si (KR); Mugong BAE, Suwon-si (KR)

(73) Assignee: SAMSUNG ELECTRONICS CO., LTD., Suwon-si (KR)

(21) Appl. No.: 19/713,044

(22) Filed: Jun. 18, 2026

Related U.S. Application Data
(63) Continuation of application No. PCT/KR2024/016425, filed on Oct. 25, 2024.

(30) Foreign Application Priority Data
Dec. 22, 2023 (KR) 10-2023-0190335

(51) Int. Cl. G10L 25/51; G10L 25/18; H04B 17/318

(52) U.S. Cl. CPC G10L 25/51; G10L 25/18; H04B 17/318

(57) ABSTRACT

An electronic device includes: memory storing at least one instruction; and at least one processor, wherein the at least one instruction, when executed by the at least one processor individually or collectively, causes the electronic device to: identify a final state of at least one user based on first state information and second state information, the first state information being obtained based on an acoustic signal pattern obtained from an acoustic signal, and the second state information being obtained based on a Wi-Fi signal pattern obtained from a Wi-Fi signal; and control the electronic device to operate in a state corresponding to the final state of the at least one user.

### CROSS-REFERENCE TO RELATED APPLICATION

This application is a continuation of International Application No. PCT/KR2024/016425, filed on Oct. 25, 2024, which is based on and claims priority to Korean Patent Application No. 10-2023-0190335, filed on Dec. 22, 2023, in the Korean Intellectual Property Office, the disclosures of which are incorporated by reference herein in their entireties.

### BACKGROUND

### 1. Field

The disclosure relates to an electronic device and an operating method thereof, and more particularly, to an electronic device operating in response to a state of a user, and an operating method thereof.

### 2. Description of Related Art

Many users tend to watch content using an electronic device such as a television (TV) before going to sleep. When the electronic device continues to output content even after the user falls asleep while watching the content, the output of the content by the electronic device actually interferes with deep sleep.

### SUMMARY

According to an aspect of the disclosure, an electronic device includes: memory storing at least one instruction; and at least one processor, wherein the at least one instruction, when executed by the at least one processor individually or collectively, causes the electronic device to: identify a final state of at least one user based on first state information and second state information, the first state information being obtained based on an acoustic signal pattern obtained from an acoustic signal, and the second state information being obtained based on a Wi-Fi signal pattern obtained from a Wi-Fi signal; and control the electronic device to operate in a state corresponding to the final state of the at least one user.

According to an aspect of the disclosure, an operating method of an electronic device, includes: identifying a final state of at least one user based on first state information and second state information, the first state information being obtained based on an acoustic signal pattern obtained from an acoustic signal, and the second state information being obtained based on a Wi-Fi signal pattern obtained from a Wi-Fi signal; and operating the electronic device in a state corresponding to the final state of the at least one user.

According to an aspect of the disclosure, a non-transitory computer-readable recording medium has recorded thereon a program that is executed by at least one processor to perform the operating method.

### BRIEF DESCRIPTION OF THE DRAWINGS

The above and other aspects, features, and advantages of certain embodiments of the present disclosure will be more apparent from the following description taken in conjunction with the accompanying drawings, in which:

FIG. 1 is a diagram illustrating that an electronic device operates in a mode corresponding to a state of a user, according to an embodiment;

FIG. 2 is a block diagram of an electronic device according to an embodiment;

FIG. 3 is a block diagram of an electronic device according to an embodiment;

FIG. 4 illustrates a Wi-Fi signal pattern obtained by a processor of FIG. 3 based on a channel state information (CSI) channel value received through a Wi-Fi signal obtaining unit, according to an embodiment;

FIG. 5 is a graph illustrating the strength of an auto correlation function (ACF) signal according to the number of users obtained based on the CSI channel value, according to an embodiment;

FIG. 6 is a graph illustrating a change in a Wi-Fi signal according to a movement of a user.

FIG. 7 is an internal block diagram of a processor according to an embodiment;

FIG. 8 is a diagram illustrating that the distance recognition unit of FIG. 7 obtains a sound source distance by using a neural network trained on distance-based acoustic data.

FIG. 9 is a table illustrating that a signal processor of FIG. 7 determines a state weight based on first state information and second state information.

FIG. 10 is a block diagram according to an example of the signal processor of FIG. 7.

FIG. 11 is a diagram illustrating separating a voice signal into voice signals for each user, according to an embodiment;

FIG. 12 is a diagram for describing that a signal separation unit of FIG. 10 identifies a Wi-Fi channel corresponding to an acoustic signal by using a neural network, according to an embodiment;

FIG. 13 is an internal block diagram of an electronic device according to an embodiment;

FIG. 14 is a flowchart illustrating an operating method of an electronic device, according to an embodiment; and

FIG. 15 is a flowchart illustrating an operating method of an electronic device, according to an embodiment.

### DETAILED DESCRIPTION

An operating method of an electronic device, according to an embodiment, may include identifying a final state of a user based on first state information obtained according to an acoustic signal pattern obtained from an acoustic signal and second state information obtained according to a Wi-Fi signal pattern obtained from a Wi-Fi signal.

In an embodiment, the operating method of the electronic device may include operating in a state corresponding to the final state of the user.

A recording medium according to an embodiment may be a computer-readable recording medium having recorded thereon a program for executing an operating method of an electronic device by a computer, the operating method including identifying a final state of a user based on first state information obtained according to an acoustic signal pattern obtained from an acoustic signal and second state information obtained according to a Wi-Fi signal pattern obtained from a Wi-Fi signal.

In an embodiment, the recording medium may be a computer-readable recording medium having recorded thereon a program capable of performing the operating method of the electronic device by a computer, the operating method including operating in a state corresponding to the final state of the user.

In the present disclosure, the expression “at least one of a, b, or c” may refer to “a”, “b”, “c”, “a and b”, “a and c”, “b and c”, or “all of a, b, and c”.

Hereinafter, embodiments of the present disclosure will be described in detail with reference to the accompanying drawings so that those skilled in the art to which the present disclosure pertains may easily carry out the present disclosure. However, the present disclosure may be implemented in various different forms and is not limited to the embodiments described herein.

The terms used in the present disclosure are described as general terms currently used in consideration of functions mentioned in the present disclosure, but may refer to various other terms according to the intention of those skilled in the art, precedents, the emergence of new technologies, and the like. Therefore, the terms used in the present disclosure should not be interpreted only by the names of the terms, but should be interpreted based on the meaning of the terms and the contents throughout the present disclosure.

In addition, the terms used in the present disclosure are used only to describe specific embodiments, and are not intended to limit the present disclosure.

Throughout the specification, when a part is said to be “connected” to another part, this includes not only a case of being “directly connected” but also a case of being “electrically connected” with another element interposed therebetween.

The use of the terms “a,” “an,” “the,” and similar referents in the context of describing the disclosure, especially in the context of the following claims, are to be construed to cover both the singular and the plural. In addition, unless there is a description explicitly specifying the order of the steps describing the method according to the present disclosure, the steps described may be performed in an appropriate order. The present disclosure is not limited to the described order of steps.

In the present specification, phrases such as “in some embodiments” or “in an embodiment” appearing in various places do not necessarily all indicate the same embodiment.

Some embodiments of the present disclosure may be described in terms of functional block components and various processing steps. Some or all of these functional blocks may be implemented by various numbers of hardware and/or software configurations that perform specific functions. For example, the functional blocks of the present disclosure may be implemented by one or more microprocessors or may be implemented by circuit components for a certain function. In addition, for example, the functional blocks of the present disclosure may be implemented in various programming or scripting languages. The functional blocks may be implemented as algorithms that are executed on one or more processors. In addition, the present disclosure may employ the related art for electronic environment setting, signal processing, and/or data processing. Terms such as “mechanism”, “element”, “means”, and “configuration” may be used widely and are not limited to mechanical and physical configurations.

In addition, connection lines or connection members between components illustrated in the drawings are merely examples of functional connections and/or physical or circuit connections. In an actual device, a connection between components may be indicated by various functional connections, physical connections, or circuit connections that are replaceable or added.

In addition, terms such as “unit” and “module” described in the specification mean a unit for processing at least one function or operation, and may be implemented in hardware or software, or a combination of hardware and software.

In addition, in the specification, the term “user” refers to a person who uses an electronic device, and may include a consumer, an evaluator, a viewer, a manager, or an installation engineer.

Hereinafter, the present disclosure will be described in detail with reference to the accompanying drawings.

The present disclosure may relate to an artificial intelligence (AI) system utilizing a machine learning algorithm and/or to an application of the AI system.

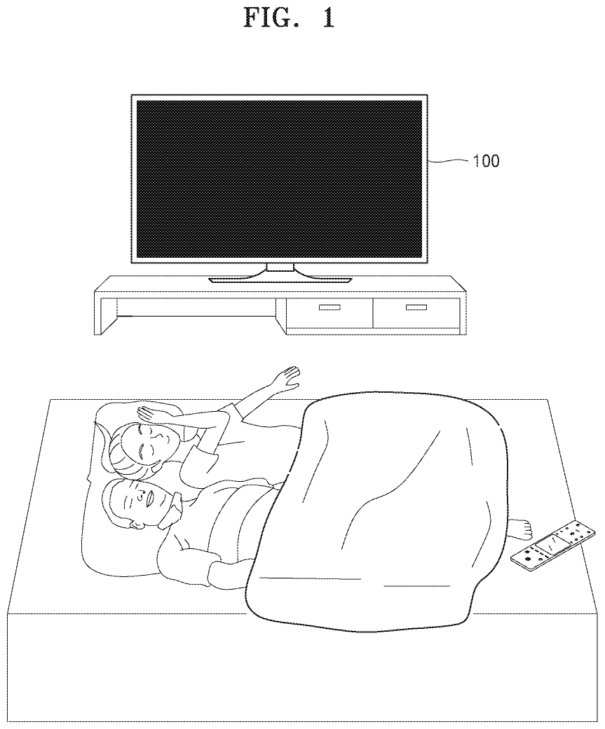

FIG. 1 is a diagram illustrating that an electronic device 100 operates in a mode corresponding to a state of a user, according to an embodiment.

In an embodiment, the electronic device 100 may be an electronic device capable of outputting content. In an embodiment, the content may be in various forms, such as video including still images or moving images, audio, subtitles, and other additional information.

In an embodiment, the electronic device 100 may be an image display device capable of outputting an image or video through a display, and/or an acoustic device capable of outputting audio. The electronic device 100 may be a fixed type or a movable type.

In an embodiment, the electronic device 100 may include at least one of a television, a desktop computer, a smartphone, a tablet personal computer (PC), a game console, an acoustic device, a mobile phone, a video phone, an e-book reader, a laptop PC, a netbook computer, a digital camera, a personal digital assistant (PDA), a portable multimedia player (PMP), a camcorder, a navigation device, a wearable device, a smart watch, a home network system, a security system, or a medical device.

When the electronic device 100 is an image display device, the electronic device 100 may be implemented not only as a flat display device, but also as a curved display device having a screen with a curvature or a flexible display device with an adjustable curvature. The output resolution of the electronic device 100 may include various resolutions, for example, high definition (HD), full HD, ultra HD, or a resolution sharper than the ultra HD.

In an embodiment, when the electronic device 100 is a television (TV), the electronic device 100 may include an operating system (OS) and a digital TV equipped with an Internet access function.

The electronic device 100 may output various types of content provided by content providers.

The content provider may refer to a terrestrial broadcasting station, a cable broadcasting station, a satellite broadcasting station, an Internet protocol television (IPTV) service provider, an over the top (OTT) service provider, or a server operator, which provides various types of content to consumers.

The content may be in various forms, such as video including still images or moving images, audio, subtitles, and other additional information.

The content may include content for viewing such as a movie or a drama, content for listening such as music, game content, content for an art work that guides or introduces an art work such as a famous painting or a statue, or the like.

In an embodiment, the electronic device 100 may receive and output various contents generated by a content provider through an external device. The external device may be implemented as various types of source devices, such as a PC, a set-top box, a Blu-ray disk player, a mobile phone, a game console, a home theater, an audio player, and a universal serial bus (USB).

The external device may be connected to the electronic device 100 through a wired communication network such as a high-definition multimedia interface (HDMI) or a wireless communication network and may provide various contents to the electronic device 100. The electronic device 100 may receive video on demand (VOD) content provided by an IPTV service provider or an OTT service provider through a set-top box and output the VOD content. The VOD service is a service that provides video desired by a user at a desired time through a communication network connection, and may include various types of content provided by an OTT service provider or an IPTV service provider. The IPTV service providers or the OTT service providers may provide real-time broadcast programs as well as VOD content.

In an embodiment, when an operating system is mounted on the electronic device 100, the electronic device 100 may stream and output various types of VOD content generated by the OTT service provider using the operating system mounted therein, in addition to the real-time broadcast program.

In an embodiment, the electronic device 100 may access the Internet and provide a web surfing service, a social network service, or the like. In addition, the electronic device 100 may perform a function of a communication center capable of checking, in real time, news, weather, e-mails, or the like.

In an embodiment, the electronic device 100 may execute various types of applications. Various types of application (applications) may be installed in the electronic device 100 by default. Alternatively, the electronic device 100 may access the Internet, search for an application requested by the user, and install the application, according to the control of the user. The electronic device 100 may provide various services by executing an application.

In an embodiment, the electronic device 100 may be wirelessly connected to an external device such as a set-top box through a wireless network conforming to a communication standard, such as Bluetooth, wireless LAN (WLAN) (Wi-Fi), wireless broadband (Wibro), world interoperability for microwave access (Wimax), CDMA, and WCDMA, and may receive an image signal from the external device.

In an embodiment, the electronic device 100 may be connected to an external device through a wired cable to receive an image signal from the external device or transmit an image signal to the external device. The wired cable may include a port capable of simultaneously transmitting a video signal and an audio signal, such as HDMI, USB, a display port (DP), Thunderbolt, or the like. Alternatively, the wired cable may include ports for separately transmitting a video signal and an audio signal.

In an embodiment, the electronic device 100 may be implemented as an electronic device that does not include an image display device such as a display. For example, when the electronic device 100 is an external device itself, such as a set-top box, a satellite broadcast receiving device, an Internet receiving device receiving content from an OTT service provider, or the like, the electronic device 100 may not include an image display device.

In this case, the electronic device 100 may be connected to the image display device in a wired manner, and may transmit a signal input from an external source to the image display device. For example, the electronic device 100 may be connected to an image display device such as a monitor or the like by using a port capable of simultaneously transmitting a video signal and an audio signal, such as USB, HDMI, DP, Thunderbolt, or the like. Alternatively, the electronic device 100 may be connected to the image display device by using a port for separately transmitting a video signal and an audio signal.

In an embodiment, the electronic device 100 may be controlled by a control device. In an embodiment, the control device may be a device, such as a remote controller, used to control the electronic device 100. The user may control various functions of the electronic device 100 using the control device.

In an embodiment, the control device may be a control device dedicated to the electronic device 100. Alternatively, in an embodiment, the control device may be an electronic device, such as a smartphone or an AI speaker, which performs an operation other than an operation of controlling the electronic device 100, as a main function. The user may control the electronic device 100 by using the control device by installing a remote controller application or the like in the control device. In this case, the control device may include at least one module capable of performing Wi-Fi, Bluetooth, or infrared communication. The control device may transmit and receive data to and from the electronic device 100 using Wi-Fi, Bluetooth, or an infrared communication module.

In an embodiment, the control device may include an input unit. The input unit may receive a user input for controlling the electronic device 100. The input unit included in the control device may include a plurality of keys. The keys may be in various forms such as a physical button for receiving a push operation by a user, a jog & shuttle, a touch button displayed on a touchpad for detecting a touch, or the like. The user may control various functions of the electronic device 100 using a plurality of keys provided in the control device.

The plurality of keys included in the control device may be used to control various functions of the electronic device 100.

In an embodiment, the electronic device 100 may obtain an acoustic signal.

In an embodiment, at least one of the electronic device 100 or the control device may include a microphone capable of receiving an acoustic signal. When a microphone is provided in the control device, the microphone may receive an analog signal, digitize the analog signal, and transmit the digitized analog signal to the electronic device 100. In an embodiment, the control device may receive the acoustic signal through the microphone and digitize the acoustic signal, and may transmit the digitized acoustic signal to the electronic device 100 by using a data transmission communication method such as Bluetooth or Wi-Fi.

In an embodiment, a plurality of microphones may be provided in the electronic device 100 to be spaced apart from each other. In an embodiment, the electronic device 100 may collect acoustic signals through a plurality of microphones and digitize the acoustic signals.

FIG. 1 illustrates a case in which the electronic device 100 is a TV, as an example.

FIG. 1 illustrates a case in which a plurality of users fall asleep while watching the electronic device 100 in the same space in which the electronic device 100 is located.

The user may use the electronic device 100 in the space where the electronic device 100 is located according to an intended purpose. For example, the user may use the electronic device 100 in a room, a living room, or an office where the electronic device 100 is located.

In an embodiment, the electronic device 100 may obtain state information about a user in a space where the electronic device 100 is located.

In an embodiment, the electronic device 100 may obtain an acoustic signal pattern from acoustic signals obtained through a plurality of microphones.

In an embodiment, the electronic device 100 may synchronize the acoustic signal with the Wi-Fi signal and obtain the acoustic signal pattern from the synchronized acoustic signal.

In an embodiment, the electronic device 100 may remove noise from the acoustic signal.

In an embodiment, when the electronic device 100 outputs the audio content through a speaker, the electronic device 100 may remove the audio content output through the speaker from the acoustic signal, together with noise.

In an embodiment, the electronic device 100 may convert a time domain-based acoustic signal, from which the audio content and the noise are filtered out, into a signal in a frequency domain.

In an embodiment, the electronic device 100 may convert the filtered acoustic signal into a Mel-spectrogram image, and obtain information about a frequency feature of a sound from the Mel-spectrogram image.

In an embodiment, the frequency feature of the sound may include at least one of pitch, timbre, or intensity of sound.

In an embodiment, the electronic device 100 may obtain an acoustic signal pattern indicating information about the frequency feature of the sound. In an embodiment, the acoustic signal pattern may include feature information indicating a pattern corresponding to a specific event. The feature information may be expressed in the form of a feature vector.

In an embodiment, the electronic device 100 may obtain state information from the acoustic signal pattern.

In an embodiment, the state information may be information indicating the state of the user.

In an embodiment, the state information may include information about at least one of whether there is a user, whether the user breathes, whether the user moves, or the number of users.

In an embodiment, the electronic device 100 may identify frequency features of various sounds generated when the user breathes, utters, or performs a specific motion by using the acoustic signal pattern.

Hereinafter, the state information obtained using the acoustic signal pattern will be referred to as first state information.

In an embodiment, the electronic device 100 may obtain a wireless fidelity (Wi-Fi) signal.

In an embodiment, the electronic device 100 may be located in a place where a Wi-Fi transmitter and a Wi-Fi receiver capable of transmitting and receiving a Wi-Fi signal are installed.

The Wi-Fi signal may use radio waves to transmit data. The radio waves may operate in a 2.4 GHZ or 5 GHz frequency band according to the Wi-Fi standard in use. A router may be connected to an Internet service provider (ISP) by wire through a DSL, a cable, an optical fiber, or the like to serve as a hub of a network. The router may convert data of a wired Internet connection into a wireless signal and broadcast the wireless signal through a specific channel in a selected frequency band.

When the router or an access point (AP) operates as a Wi-Fi transmitter and transmits a Wi-Fi signal, a Wi-Fi receiver or a Wi-Fi sensor may receive the Wi-Fi signal.

In an embodiment, the electronic device 100 may obtain a Wi-Fi signal through a Wi-Fi receiver.

In an embodiment, the electronic device 100 may synchronize the acoustic signal and the Wi-Fi signal and obtain a Wi-Fi signal pattern from the synchronized Wi-Fi signal.

In an embodiment, the electronic device 100 may collect a pattern trend of a Wi-Fi signal for a certain period of time and identify a pattern corresponding to a specific event therefrom.

The Wi-Fi signal may be transmitted indoors as a multipath signal through reflection, scattering, and diffraction. Wi-Fi signals have a characteristic of changing signal strength when reflected by an object or passing near an object. Accordingly, a signal variation range between channels or a signal strength varies according to the presence or absence of a user in a certain space or the number of users in a space.

In an embodiment, the electronic device 100 may identify the state information by using a signal pattern that varies according to the reflection/diffraction of a Wi-Fi signal.

Hereinafter, the state information obtained from the Wi-Fi signal will be referred to as second state information.

In an embodiment, the electronic device 100 may identify the final state of a user based on the first state information and the second state information.

In some cases, the acoustic signal obtained by the electronic device 100 through a plurality of microphones may include an acoustic signal generated from the outside, not in the space where the electronic device 100 is located.

In addition, the Wi-Fi signal obtained by the electronic device 100 may be a signal affected by a user outside the space where the electronic device 100 is located.

The Wi-Fi signal, which has high sensitivity and can operate even under a non-line-of-sight (NLOS) condition, may be sensed even by a user at a long distance and with a wide angle range. Accordingly, the Wi-Fi signal may be affected by the presence or absence of another user or the motions of another user in a neighbor's house not in the same household.

In an embodiment, the electronic device 100 may obtain an acoustic distance for the acoustic signal to identify whether the state information is state information about the user in the space where the electronic device 100 is located. In an embodiment, the acoustic distance may be a distance from a location where the acoustic signal is generated to the electronic device 100. Specifically, the acoustic distance may be a distance from a location where the acoustic signal is generated to a plurality of microphones provided in the electronic device 100.

In an embodiment, the electronic device 100 may measure the distance from the location where the acoustic signal is generated to the microphone in consideration of an interval between the plurality of microphones, the speed of sound, and a difference in time at which each microphone collects the acoustic signal.

In an embodiment, the electronic device 100 may identify whether the acoustic distance is within a predetermined range. The preset range may be information indicating a range of a certain preset distance. The electronic device 100 may identify whether the distance between the electronic device 100 and an acoustic signal generation point, that is, the acoustic distance, is included in a range of a certain distance.

In an embodiment, when the acoustic distance is within a preset range, the electronic device 100 may determine the final state of the user based on the first state information and the second state information.

In an embodiment, the electronic device 100 may detect a plurality of users based on at least one of the Wi-Fi signal or the acoustic signal.

In an embodiment, the electronic device 100 may identify whether there are a plurality of users based on at least one of the variation range or the signal strength of the Wi-Fi signal.

In an embodiment, the electronic device 100 may detect a plurality of users based on at least one of a voice event or a breathing event being detected in the acoustic signal.

In FIG. 1, the electronic device 100 may identify that there are a plurality of users based on the strength of the Wi-Fi signal or a user's breathing event.

In an embodiment, when detecting a plurality of users, the electronic device 100 may identify a signal corresponding to each of the plurality of users from the Wi-Fi signal and the acoustic signal.

In an embodiment, the electronic device 100 may identify an acoustic signal of each of a plurality of users from an acoustic signal collected by using a plurality of microphones.

In an embodiment, the electronic device 100 may identify an acoustic signal generated due to an utterance feature or a breathing feature of each user or a motion of each user from the acoustic signal by separating the acoustic signal from an acoustic signal of another user.

In an embodiment, the electronic device 100 may obtain the first state information for each user from the separated acoustic signal.

In FIG. 1, the electronic device 100 may obtain first state information for each of two users.

In an embodiment, the electronic device 100 may identify a Wi-Fi channel corresponding to an acoustic signal separated for each user in response to the detection of a plurality of users.

In an embodiment, the electronic device 100 may identify a Wi-Fi channel corresponding to the acoustic signal of each user by using a neural network previously trained on a relationship between the acoustic signal and the Wi-Fi signal.

In an embodiment, the electronic device 100 may obtain the second state information for each user from the identified Wi-Fi channel.

In FIG. 1, the electronic device 100 may identify a Wi-Fi channel corresponding to each of two users based on an acoustic signal corresponding to each of the two users.

In an embodiment, the electronic device 100 may determine a state weight of each user by using the first state information and the second state information corresponding to each user.

For example, in FIG. 1, the electronic device 100 may determine a state weight of a first user using the first state information and the second state information corresponding to the first user among the two users, and may determine a state weight of a second user using the first state information and the second state information corresponding to the second user.

In an embodiment, the electronic device 100 may accumulate the state weight of each user for a certain period of time, and identify the final state of each user therefrom.

In an embodiment, the final state may refer to a final state of the user determined by considering the first state information based on the acoustic signal and the second state information based on the Wi-Fi signal for a certain period of time.

In FIG. 1, the electronic device 100 may determine that the final state of each of the two users is a sleep state.

In an embodiment, the electronic device 100 may operate in a mode corresponding to the final state of the user. In an embodiment, the mode may refer to a state in which a specific motion or task may be performed. In the present disclosure, the mode may also be referred to as a state.

In an embodiment, when there are a plurality of users and it is determined that all of the plurality of users are in a sleep state, the electronic device 100 may operate in a sleep mode in response thereto.

In an embodiment, the sleep mode may be referred to as a sleep state, and may be a low power mode/state in which power of the electronic device 100 is saved.

Unlike the sleep mode, a normal mode may be a mode in which power is supplied to all components of the electronic device 100. The normal mode may be referred to as a normal state.

In an embodiment, while the electronic device 100 is operating in the normal mode, when one user is detected and it is determined that the final state of the user is the sleep state, the electronic device 100 may operate by changing the mode/state from the normal mode to the sleep mode.

In an embodiment, while the electronic device 100 is operating in the normal mode, when a plurality of users are detected and it is determined that the final states of the plurality of users are all the sleep states, the electronic device 100 may operate by changing the mode from the normal mode to the sleep mode.

In an embodiment, when it is determined that the final state of all the detected users is the sleep state, the electronic device 100 may turn off the functions, leaving only the function of processing the Wi-Fi signal and the acoustic signal. The electronic device 100 may prevent any more video content from being output through the screen and prevent any more audio content from being output through the speaker.

In FIG. 1, the electronic device 100 may change the operation mode of the electronic device 100 to the sleep mode, in response to the final states of the two users both being the sleep state.

As such, according to an embodiment, the electronic device 100 may more accurately identify the state of a user by using the Wi-Fi signal and the acoustic signal together.

In addition, according to an embodiment, the electronic device 100 may accurately identify the state of a plurality of users not only when there is one user, but also when there are a plurality of users, by using the Wi-Fi signal and the acoustic signal together.

In addition, according to an embodiment, when the user falls asleep while watching the electronic device 100, the sleep state of the user is identified, and the content output is stopped in response to the sleep state, thereby helping the user to sleep well.

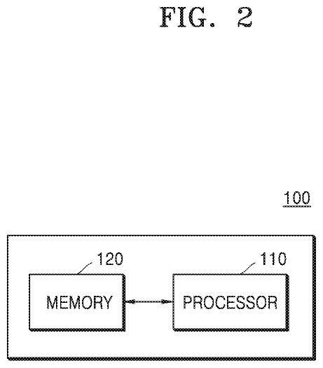

FIG. 2 is a block diagram of the electronic device 100 according to an embodiment.

The electronic device 100 of FIG. 2 may be an example of the electronic device 100 described with reference to FIG. 1.

Referring to FIG. 2, the electronic device 100 may include a memory 120 and a processor 110.

In an embodiment, the memory 120 may store at least one instruction. The memory 120 may store at least one program executed by the processor 110. In addition, the memory 120 may store data input to the electronic device 100 or output from the electronic device 100.

The memory 120 may include at least one type of storage media among a flash memory type, a hard disk type, a multimedia card micro type, a card type memory (e.g., SD or XD memory), random access memory (RAM), static random access memory (SRAM), read-only memory (ROM), electrically erasable programmable read-only memory (EEPROM), programmable read-only memory (PROM), a magnetic memory, a magnetic disk, and an optical disk.

In an embodiment, the memory 120 may store one or more instructions for obtaining the first state information from the acoustic signal.

In an embodiment, one or more instructions for outputting audio content may be stored in the memory 120.

In an embodiment, the memory 120 may store one or more instructions for filtering audio content and noise from the acoustic signal.

In an embodiment, the memory 120 may store one or more instructions for converting the filtered acoustic signal into a Mel-spectrogram image.

In an embodiment, the memory 120 may store one or more instructions for obtaining first state information from a frequency feature of a sound obtained from the Mel-spectrogram image.

In an embodiment, the frequency feature of the sound may include at least one of pitch, timbre, or intensity of sound.

In an embodiment, the memory 120 may store one or more instructions for obtaining the second state information from the Wi-Fi signal.

In an embodiment, the memory 120 may store one or more instructions for obtaining a Wi-Fi signal pattern from the Wi-Fi signal.

In an embodiment, the memory 120 may store one or more instructions for obtaining second state information from a pattern trend of the Wi-Fi signal for a certain period of time.

In an embodiment, the memory 120 may store one or more instructions for identifying the final state of a user based on the first state information and the second state information.

In an embodiment, a preset range may be stored in the memory 120.

In an embodiment, the memory 120 may store one or more instructions for identifying whether a distance to the acoustic signal generation point is within the preset range.

In an embodiment, the memory 120 may store one or more instructions for detecting a plurality of users based on at least one of the acoustic signal or the Wi-Fi signal.

In an embodiment, the memory 120 may store one or more instructions for detecting at least one of a voice event of a plurality of users or a breathing event of the plurality of users in the acoustic signal.

In an embodiment, the memory 120 may store one or more instructions for detecting a plurality of users based on at least one of the variation range or the signal strength of the Wi-Fi signal.

In an embodiment, the memory 120 may store one or more instructions for identifying a corresponding signal for each user from the acoustic signal and the Wi-Fi signal in response to the detection of a plurality of users.

In an embodiment, the memory 120 may store one or more instructions for separating the first acoustic signal including the utterance feature of the first user from the acoustic signal collected using the plurality of microphones.

In an embodiment, the memory 120 may store one or more instructions for obtaining the first state information corresponding to the first user from the first acoustic signal.

In an embodiment, the memory 120 may store one or more instructions for identifying a first Wi-Fi channel corresponding to the first acoustic signal by using a neural network trained on a relationship between the acoustic signals and the Wi-Fi signals.

In an embodiment, the memory 120 may store one or more instructions for obtaining the second state information corresponding to the first user from the first Wi-Fi channel.

In an embodiment, the memory 120 may store one or more instructions for determining the state weight of the first user by using the first state information and the second state information corresponding to the first user.

In an embodiment, the memory 120 may store one or more instructions for identifying a final state of the first user based on a state weight of the first user obtained over a predetermined time.

In an embodiment, one or more instructions for operating in a mode corresponding to the final state of a user may be stored in the memory 120.

In an embodiment, when it is identified that the final state of all the detected users is the sleep state, the memory 120 may store one or more instructions for operating in the sleep mode.

The processor 110 according to an embodiment controls the overall operation of the electronic device 100. The processor 110 according to an embodiment may perform a function of controlling a signal flow between internal components of the electronic device 100 and processing data.

In an embodiment, the processor 110 may control the electronic device 100 to function by executing the one or more instructions stored in the memory 120.

In an embodiment, the processor 110 may include a single core, a dual core, a triple core, a quad core, and a multiple core thereof.

In an embodiment, the processor 110 may include one or a plurality of processors. For example, the processor 110 may include a plurality of processors. In this case, the processor 110 may be implemented by a main processor and a sub processor.

In addition, the processor 110 may include at least one of a central processing unit (CPU), a graphics processing unit (GPU), or a video processing unit (VPU). Alternatively, in an embodiment, the processor 110 may be implemented in the form of a system on chip (SoC) in which at least one of CPU, GPU, or VPU is integrated. Alternatively, in an embodiment, the processor 110 may further include a neural processing unit (NPU).

In an embodiment, the at least one processor 110 may obtain the first state information from the acoustic signal.

In an embodiment, the at least one processor 110 may output audio content through the speaker provided in the electronic device 100.

In an embodiment, the at least one processor 110 may filter audio content and noise from the acoustic signal.

In an embodiment, the at least one processor 110 may convert the filtered acoustic signal into a Mel-spectrogram image.

In an embodiment, the at least one processor 110 may obtain the first state information from the frequency feature of the sound obtained from the Mel-spectrogram image.

In an embodiment, the at least one processor 110 may obtain the second state information from the Wi-Fi signal.

In an embodiment, the at least one processor 110 may obtain a Wi-Fi signal pattern from the Wi-Fi signal.

In an embodiment, the at least one processor 110 may obtain the second state information from a pattern trend of the Wi-Fi signal for a predetermined time.

In an embodiment, the at least one processor 110 may perform synchronization between the acoustic signal and the Wi-Fi signal.

In an embodiment, the at least one processor 110 may synchronize the acoustic signal and the Wi-Fi signal by using an interval time between two signals indicating an input time difference between the acoustic signal and the Wi-Fi signal.

In an embodiment, the at least one processor 110 may obtain the first state information and the second state information from the synchronized acoustic signal and Wi-Fi signal, respectively.

In an embodiment, the at least one processor 110 may identify the state of a user based on the first state information and the second state information.

In an embodiment, the at least one processor 110 may identify whether the distance to the acoustic signal generation point is within a preset range.

In an embodiment, the at least one processor 110 may detect a plurality of users based on at least one of the acoustic signal or the Wi-Fi signal.

In an embodiment, the at least one processor 110 may detect at least one of a voice event of a plurality of users or a breathing event of the plurality of users in the acoustic signal.

In an embodiment, the at least one processor 110 may detect a plurality of users based on at least one of the variation range or the signal strength of the Wi-Fi signal.

In an embodiment, the at least one processor 110 may identify a corresponding signal for each user from the acoustic signal and the Wi-Fi signal in response to the detection of the plurality of users.

In an embodiment, the at least one processor 110 may separate the acoustic signal including the utterance feature of the first user from the acoustic signal collected by using the plurality of microphones.

In an embodiment, the at least one processor 110 may obtain first state information corresponding to the first user from the first acoustic signal.

In an embodiment, the at least one processor 110 may identify the first Wi-Fi channel corresponding to the first acoustic signal by using a neural network trained on the relationship between the acoustic signal and the Wi-Fi signal.

In an embodiment, the at least one processor 110 may obtain second state information corresponding to the first user from the first Wi-Fi channel.

In an embodiment, the at least one processor 110 may determine the state weight of the first user by using the first state information and the second state information corresponding to the first user.

In an embodiment, the at least one processor 110 may identify the final state of the first user, based on the state weight of the first user obtained for a predetermined time.

In an embodiment, the at least one processor 110 may operate in a mode/state corresponding to the final state of a user.

In an embodiment, when it is identified that all of the detected users are in the sleep state, the at least one processor 110 may operate in the sleep state.

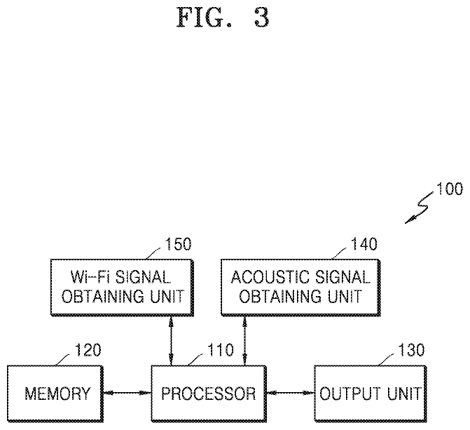

FIG. 3 is a block diagram of the electronic device 100 according to an embodiment.

The electronic device 100 of FIG. 3 may be an example of the electronic device 100 described with reference to FIG. 2.

Hereinafter, redundant descriptions of those described with reference to FIG. 2 are omitted.

Referring to FIG. 3, the electronic device 100 may further include an output unit 130, an acoustic signal obtaining unit 140, and a Wi-Fi signal obtaining unit 150, in addition to the memory 120 and the processor 110.

The output unit 130 according to an embodiment may output content. The output unit 130 may include at least one of a display for outputting video content or a speaker for outputting audio content.

The output unit 130 may output video and/or audio content while the electronic device 100 operates in the normal mode.

The acoustic signal obtaining unit 140 according to an embodiment may collect an acoustic signal. The acoustic signal may also be referred to as an audio signal.

The acoustic signal obtaining unit 140 may include a microphone array. The acoustic signal obtaining unit 140 may collect an audio signal through the microphone array.

The microphone array may collect audio signals including audio content output through the speaker, a user's voice, various sounds made by a user, noise, or the like, and digitize analog audio signals.

In an embodiment, the processor 110 may separate noise from the audio signal collected through the acoustic signal obtaining unit 140. For example, the acoustic signal obtaining unit 140 may separate noise from the audio signal using a convolutional neural network (CNN) model such as Wave-U-NET, but is not limited thereto.

In an embodiment, the processor 110 may separate the audio content output through the speaker from the audio signal collected through the acoustic signal obtaining unit 140. In an embodiment, the processor 110 may capture audio content immediately before being output through the speaker included in the output interface 130. The processor 110 may synchronize the captured audio content with the audio signal collected through the microphone array included in the acoustic signal obtaining unit 140 and then remove the captured audio content from the audio signal.

In an embodiment, the processor 110 may filter the audio content and the noise from the acoustic signal and then convert a time domain-based acoustic signal into a signal in a frequency domain. For example, the processor 110 may convert the acoustic signal into a Mel-spectrogram image.

In an embodiment, the processor 110 may obtain a frequency feature of a sound from the Mel-spectrogram image. The frequency feature of the sound may include at least one of pitch, timbre, or intensity of the sound.

In an embodiment, the processor 110 may obtain the first state information from the frequency feature of the sound.

In an embodiment, the processor 110 may identify the state of the user corresponding to the frequency feature of the sound obtained from the current acoustic signal by using a neural network previously trained on a feature vector representing the frequency features of various sounds.

For example, the processor 110 may input a currently obtained acoustic signal pattern to a neural network previously trained on feature vectors of various acoustic signals, such as a user's breathing sound, a footstep sound, a food eating sound, and a snack bag sound, and obtain state information corresponding to the acoustic signal pattern from the neural network.

The Wi-Fi signal obtaining unit 150 according to an embodiment may obtain a Wi-Fi signal. An indicator for a channel that may be measured by the Wi-Fi receiver includes received signal strength indicator (RSSI), channel state information (CSI), or the like. For convenience of description, hereinafter, a case in which the Wi-Fi signal obtaining unit 150 obtains data in the form of CSI is described, but the present disclosure is not limited thereto.

In an embodiment, the Wi-Fi signal obtaining unit 150 may receive a Wi-Fi CSI channel value. The CSI is information indicating a channel frequency response characteristic of a multipath signal. The CSI is a sampled value of a channel frequency response (CFR) and indicates a frequency domain value of a channel impulse response (CIR). The Wi-Fi signal obtaining unit 150 may receive CIS raw data in real time.

The Wi-Fi signal is transmitted indoors as a multipath signal through reflection, scattering, and diffraction. The Wi-Fi CSI includes an amplitude, a phase, and a delay time of each multipath component. The Wi-Fi signal transmitted to the receiver in the indoor environment may be expressed by the amplitude, phase, and delay time of the multipath component.

In an embodiment, the processor 110 may preprocess CIS raw data received through the Wi-Fi signal obtaining unit 150. For example, the processor 110 may obtain an average value between a plurality of channels by grouping adjacent channels, except for a null channel among the Wi-Fi signals.

In an embodiment, the processor 110 may group adjacent channels of subcarriers into a predetermined number of groups, and obtain an average value of a plurality of channel signals included in each group. For example, the processor 110 may group 256 subcarriers into seven channels so as to be divided into a total of 36 groups.

In an embodiment, the processor 110 may obtain an average value of seven channel signals included in each group, and obtain the average value for a predetermined time, for example, 25 seconds, to obtain a time-series pattern trend of data. In an embodiment, the processor 110 may obtain the second state information from the time-series pattern trend of the Wi-Fi data.

In an embodiment, the processor 110 may determine the final state of the user based on the first state information and the second state information.

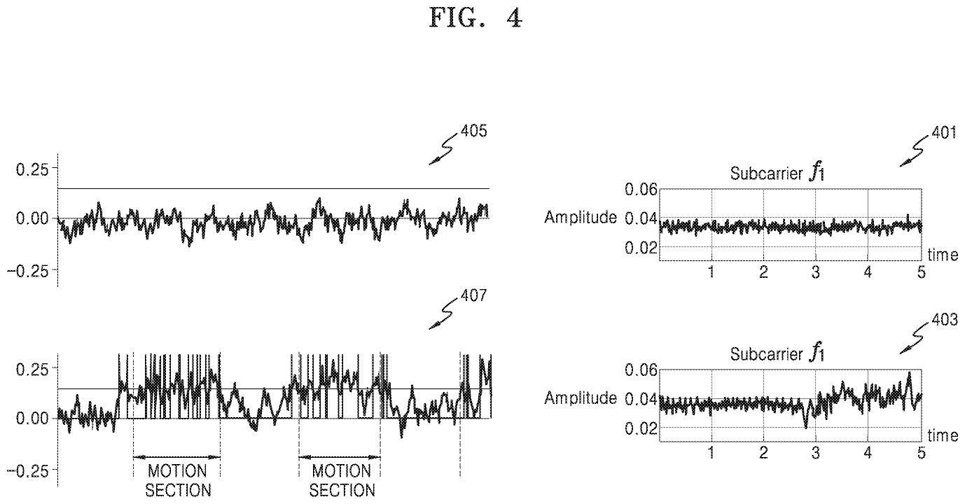

FIG. 4 illustrates a Wi-Fi signal pattern obtained by the processor 110 of FIG. 3 based on a CSI channel value input through the Wi-Fi signal obtaining unit 150, according to an embodiment.

The reflection/diffraction of the Wi-Fi signal affects the CSI raw value. Accordingly, the Wi-Fi signal pattern varies according to the presence or absence of a user, the number of users, the degree of breathing of a user, the movement of a user, or the like.

In FIG. 4, graphs 401 and 403 are graphs showing the amplitude of a subcarrier over time. Reference numeral 401 indicates an amplitude of a subcarrier when the user does not move, and reference numeral 403 indicates that the amplitude of the subcarrier varies according to the movement of the user when the user remains stationary and then starts moving after a predetermined time.

It can be seen that the degree of reflection/diffraction of the Wi-Fi signal varies according to the presence or absence of the user's movement, and accordingly, the CSI raw value is affected and the subcarrier value varies.

In an embodiment, the processor 110 may interpret a signal obtained through the Wi-Fi signal obtaining unit 150 by an auto correlation function (ACF). The processor 110 may identify a signal change between the two transceivers by examining similarity between a signal transmitted from the AP and a signal received after being reflected by an object. When a change (diffraction) between two signals at time points x1 and x2 over time is large, the ACF is close to the maximum value, and when the change of the signal is small, the ACF value is close to the minimum value.

In FIG. 4, graphs 405 and 407 are graphs showing ACF changes over time. Reference numeral 405 indicates an ACF change when the user is not in a space, and reference numeral 407 indicates an ACF change that varies according to movement and stopping when the user repeats the movement and stopping.

Referring to the graphs 405 and 407, it can be seen that the signal between the transmitter and the receiver varies according to the presence or absence of the user and the movement of the user. Accordingly, it is possible to identify the presence or absence of the user and the movement of the user through the ACF.

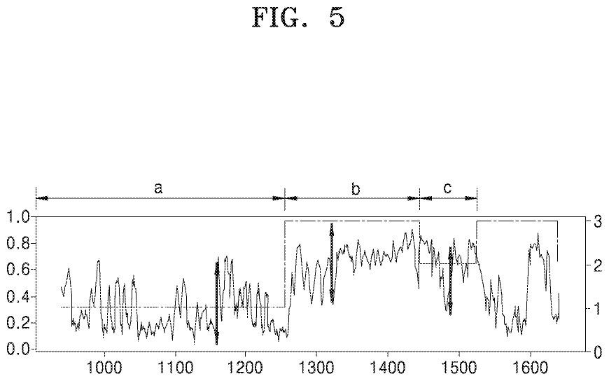

FIG. 5 is a graph illustrating the strength of an ACF signal according to the number of users obtained based on a CSI channel value, according to an embodiment.

As the number of people in a limited space such as a room, a living room, or an office increases, the reflection or diffraction of Wi-Fi signals occurs more. The CSI raw value varies according to the reflection or diffraction of the Wi-Fi signal, and thus, the signal variation range between channels or the signal strength varies.

The graph shown in FIG. 5 shows that the strength of the ACF signal varies based on the number of users. In the graph of FIG. 5, section a represents the ACF signal when there is one user, and section b represents the ACF signal when there are three users. In the graph of FIG. 5, a section c represents the ACF signal when there are two users. As illustrated in the graph of FIG. 5, it can be seen that the maximum value and the minimum value of the strength of the ACF signal are different in sections a, b, and c. That is, it can be seen that the minimum value and the maximum value of the strength of the ACF signal are large in the order of section b, section c, and section a. This may mean that the strength of the ACF signal increases as the number of users increases.

In an embodiment, the processor 110 may obtain the state information from the Wi-Fi signal by using a neural network.

In an embodiment, the neural network used by the processor 110 may be a deep learning model trained on a change in a Wi-Fi signal pattern that varies based on the presence or absence of a user, the number of users, a movement event of a user, or the like.

In an embodiment, the processor 110 may input the currently obtained Wi-Fi signal or the pattern of the Wi-Fi signal to the neural network that has been trained, and may obtain state information corresponding to the pattern of the Wi-Fi signal from the neural network.

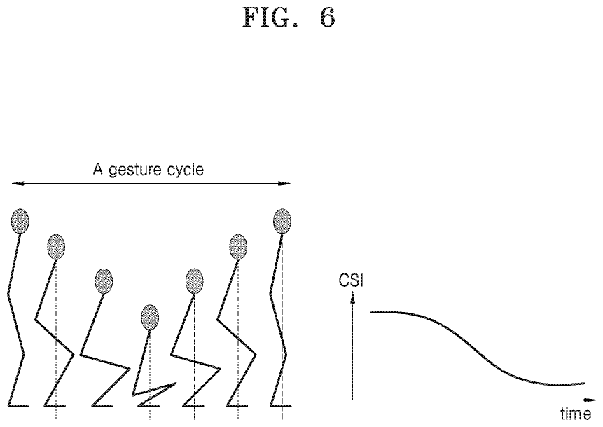

FIG. 6 is a graph illustrating a change in a Wi-Fi signal based on a movement of a user.

A signal pattern changes based on the reflection/diffraction of the Wi-Fi signal. For example, the Wi-Fi signal also has a different pattern based on a change in the movement of the chest when a user deeply inhales and exhales.

Referring to FIG. 6, when a user performs a specific motion for a predetermined time, for example, when a standing user sits down and then stands up again, it can be seen that the Wi-Fi CSI raw data is obtained as a signal having a pattern of a predetermined form.

In an embodiment, the processor 110 may identify what action the user performs, by using such a Wi-Fi signal pattern.

In an embodiment, the processor 110 may detect various events, such as whether the user inhales, exhales, takes a deep breath, takes a light breath, is sitting, stands up and moves, is sitting and remains stationary, or takes a deep breath while sitting still, using the Wi-Fi signal pattern.

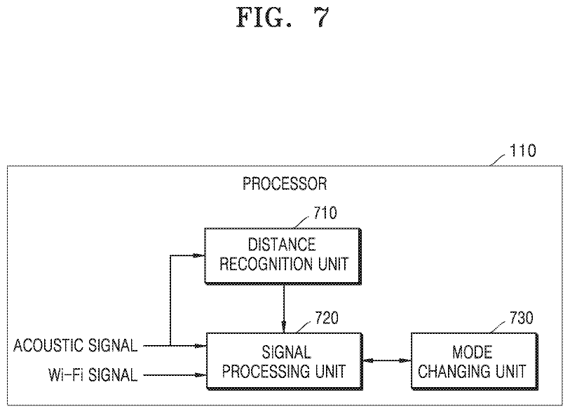

FIG. 7 is an internal block diagram of the processor 110 according to an embodiment.

The processor 110 of FIG. 7 may be an example of the processor 110 of FIG. 2 or 3.

Referring to FIG. 7, the processor 110 may include a distance recognition unit 710, a signal processing unit 720, and a mode changing unit 730.

In an embodiment, the component included in the processor 110 may be a module. In an embodiment, the module may mean a functional and structural combination of hardware for performing the technical concept of the present disclosure and software for driving the hardware. For example, the module may mean a logical unit of a predetermined code and a hardware resource for performing the predetermined code, and does not necessarily mean a physically connected code or is not limited to one type of hardware.

As the Wi-Fi signal may pass through a wall or an obstacle and has high sensitivity, even a user outside a space where the electronic device 100 is located may be detected. In this case, there may be a problem in that the electronic device 100 incorrectly changes the mode based on the Wi-Fi signal affected by the user who is not in the same space in which the electronic device 100 is located.

In an embodiment, in order to prevent such a problem, the electronic device 100 may identify the distance at which the acoustic signal is generated by using the acoustic signal.

The distance recognition unit 710 according to an embodiment may be a module for identifying whether the acoustic distance is within a preset range.

The preset range may also be referred to as a limit distance range.

The preset range may be used to identify whether the distance from the electronic device 100 to the acoustic signal generation point is within a certain range. The preset range may be pre-stored in the electronic device 100 as a default value.

Alternatively, in an embodiment, the electronic device 100 may set a range based on each place.

In an embodiment, the electronic device 100 may output an interface screen including content guiding a user to utter a specific sound for a predetermined time to set the limit distance range. For example, the interface screen may include a text signal with content such as “Please utter the sound “Ah~” for 5 seconds”.

In an embodiment, the electronic device 100 may output an audio guide signal including content to utter a specific sound.

When the interface screen or the audio signal indicating to utter a specific sound for a predetermined time, the user may correspondingly utter the specific sound for the predetermined time. The user may utter a specific sound for a predetermined time in a place where the electronic device 100 is mainly used. For example, when the user mainly watches the electronic device 100 on the sofa of the living room, the user may sit on the sofa and utter a specific sound.

In an embodiment, when the user utters a specific sound for a predetermined time, the electronic device 100 may obtain a voice signal of the user. The electronic device 100 may collect a user's utterance through the microphone array provided in the electronic device 100 and measure a distance from the user therefrom.

When a microphone is embedded in the control device for controlling the electronic device 100, the user may press a voice input button provided in the control device and then utter a specific voice. The microphone provided in the control device may receive the user's utterance as an analog signal, digitize the received signal, and transmit the digitized signal to the electronic device 100.

In an embodiment, the electronic device 100 may measure a distance from the electronic device 100 to the user and then set a limit distance range by applying an error range to the distance to the user. For example, when the distance from the electronic device 100 to the user is 3 m and the error range is 20 cm, the electronic device 100 may set, as the limit distance range, a range from 280 cm obtained by subtracting the error range from the distance to the user to 320 cm obtained by adding the error range to the distance.

In an embodiment, the electronic device 100 may store the limit distance range as a preset range and then determine whether the distance to the acoustic signal is within the preset range.

In an embodiment, when the acoustic signal is input, the distance recognition unit 710 may obtain an acoustic distance and identify whether the acoustic distance is within a preset range.

In an embodiment, the distance recognition unit 710 may measure a distance from a location where the acoustic signal is generated to the electronic device 100.

In an embodiment, the distance recognition unit 710 may estimate a distance and a direction in which an acoustic signal is generated, by using a feature of the microphone array.

As an example, the distance recognition unit 710 may obtain a phase difference of the acoustic signal reaching the plurality of different microphones, and obtain the acoustic distance from the microphone to a sound source by using the speed of sound and a time difference at which the acoustic signal reaches the plurality of microphones.

The distance recognition unit 710 may measure an acoustic distance from a location where an acoustic signal is generated to a microphone by using a time difference at which an acoustic signal generated at the same location reaches a plurality of microphone arrays, the speed of sound, an interval between the plurality of microphone arrays, that is, a distance and an angle between the plurality of microphone arrays, or the like.

In an embodiment, the distance recognition unit 710 may measure the distance between the user and the electronic device 100 by using an ultrasonic sensor. For example, when an ultrasonic sensor is attached to a control device controlling the electronic device 100, the electronic device 100 may receive an ultrasonic signal from the control device and obtain a distance between the control device and the electronic device 100 based on the ultrasonic signal.

In an embodiment, the distance recognition unit 710 may obtain an acoustic distance by using a neural network trained on distance-based acoustic data.

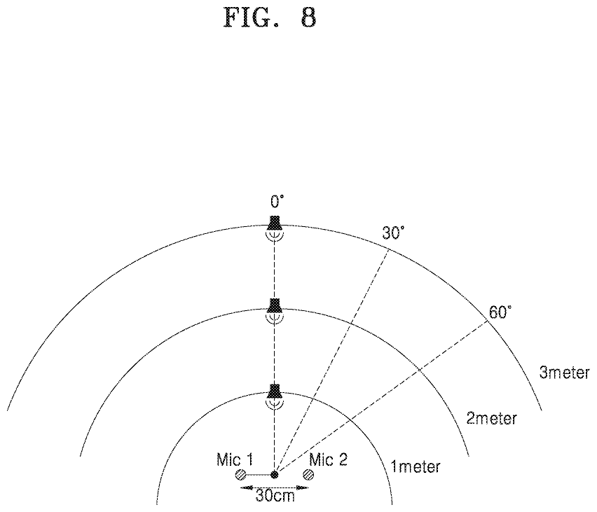

Hereinafter, an example in which the distance recognition unit 710 obtains an acoustic distance by using a neural network trained on the distance-based acoustic data will be described with reference to FIG. 8.

FIG. 8 is a diagram illustrating that the distance recognition unit 710 of FIG. 7 obtains an acoustic distance by using a neural network trained on the distance-based acoustic data.

In an embodiment, when the same sound source is located at different distances and angles, the manufacturer may obtain features of sounds input to a plurality of microphones and train the neural network by using the features as training data.

As illustrated in FIG. 8, for example, it is assumed that two microphones Mic 1 and Mic 2 are provided in the electronic device 100 to be spaced apart from each other by a predetermined distance. A manufacturer that manufactures the electronic device 100 may position the same sound source at a location at which distances from the center point between the two microphones provided in the electronic device 100 are 1 meter, 2 meters, and 3 meters, respectively, and at locations at which azimuth angles from the center point between the two microphones are 0 degrees, 30 degrees, and 60 degrees, respectively.

The plurality of microphones provided in the electronic device 100 may collect acoustic signals. The acoustic signals obtained by the plurality of microphones may include background noise or the like. After removing noise from the acoustic signal, the manufacturer may obtain feature information in the form of a feature vector from the acoustic signal from which noise has been removed.

The manufacturer may obtain, as the feature information, at least one of various parameters obtained through digitization, frequency conversion, or the like of the acoustic signal, including pitch, formant, linear predictive cepstral coefficient (LPCC), Mel-frequency cepstral coefficient (MFCC), perceptual linear predictive (PLP), or the like of the acoustic signal. For example, the manufacturer may obtain the feature information from the voice using the MFCC algorithm. The MFCC algorithm is a technique of dividing an acoustic signal into small frames of about 20 ms-40 ms and analyzing a spectrum of the divided frames to extract features.

In an embodiment, the manufacturer may train the neural network by using the feature information obtained from the acoustic signal together with information on the location of the acoustic signal as training data.

That is, in an embodiment, when a specific sound source for each distance/location is generated, the manufacturer may convert an acoustic signal received by the microphone for each channel into a Mel-spectrogram image, and train the neural network by using the feature of the corresponding sound source, as training data, together with the distance or angle label.

In an embodiment, the neural network may be trained on a distance-related feature, for example, a time delay or sensitivity, together with a distance or an angle label from a signal in a frequency domain, and may be trained on how the feature of a sound varies according to the distance and/or angle, how the feature of a sound input to a plurality of microphones differs when the plurality of microphones receive a sound generated at the same position, or the like.

In an embodiment, the neural network trained on the distance-based acoustic data may be a CNN-based deep learning model. In an embodiment, the neural network trained on the distance-based acoustic data may be a neural network trained to extract frequency-based information from each input spectrogram through convolution and obtain an acoustic distance from feature information obtained from an acoustic signal converted into a frequency domain.

In an embodiment, the distance recognition unit 710 may infer a distance at which audio is generated from an acoustic signal by using a neural network.

Referring back to FIG. 7, when the acoustic distance is within the limit distance range, the distance recognition unit 710 may notify the signal processing unit 720 of the acoustic distance.

The signal processing unit 720 according to an embodiment may be a module that receives an audio signal and a Wi-Fi signal and obtains state information from the audio signal and the Wi-Fi signal.

In an embodiment, when the acoustic signal is obtained, the signal processing unit 720 may identify whether the acoustic signal is an acoustic signal within the limit distance range. When the acoustic signal is an acoustic signal within the limit distance range, the signal processing unit 720 may obtain first state information from the acoustic signal.

In an embodiment, the signal processing unit 720 may obtain the second state information from the Wi-Fi signal.

In an embodiment, the signal processing unit 720 may identify the state of a user based on the first state information and the second state information.

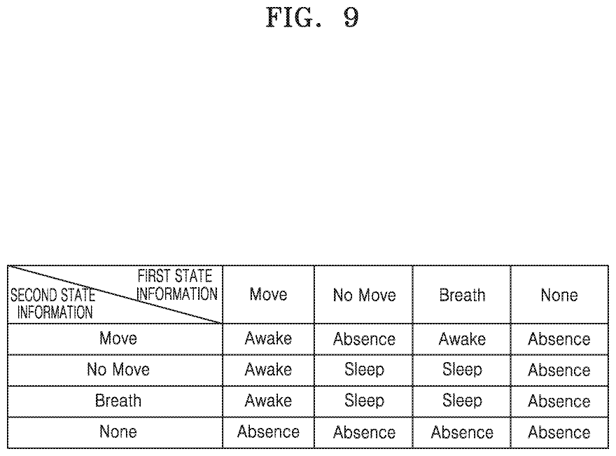

FIG. 9 is a table illustrating that the signal processing unit 720 of FIG. 7 determines a state weight based on the first state information and the second state information.

Referring to FIG. 9, the signal processing unit 720 may obtain the first state information by using an acoustic signal pattern obtained from an acoustic signal. The first state information may indicate whether the user moves, whether the user breathes, whether the user is absent, or the like.

The signal processing unit 720 may obtain the second state information using a Wi-Fi signal pattern obtained from the Wi-Fi signal. The second state information may also indicate whether the user is moving, whether the user is breathing, whether the user is absent, or the like.

In an embodiment, the signal processing unit 720 may determine the state weight of the user by using both the first state information and the second state information.

In an embodiment, the state weight may be a weight or importance assigned to each individual state to determine the final state of a user. In an embodiment, the individual state may include a user absence state, a user sleep state, a user awake state, or the like.

In an embodiment, as shown in the table of FIG. 9, the signal processing unit 720 may store a table or a matrix including the first state information and the second state information in rows and columns. Each component of the matrix may be a state weight determined in consideration of both the first state information and the second state information.

Referring to the table of FIG. 9, when the first state information is the movement state (Move), a weight may be assigned to the awake state (Awake) except when the second state information is the user absence state (None).

The Wi-Fi signal has a signal pattern characteristics that is clearly distinguished based on the presence or absence of a user. Accordingly, even when the first state information obtained from the acoustic signal is a movement state, when the second state information obtained from the Wi-Fi signal indicates a user absence state, a weight may be assigned to the user absence state.

As the Wi-Fi signal is likely to detect a user outside the limit distance range, even when the second state information is the movement state, when the first state information based on the acoustic signal obtained within the limit distance range is a state in which there is no movement, a weight may be assigned to the user absence state.

For the same reason, when the first state information is a user absence state, a weight may be assigned to the user absence state regardless of the second state information.

When the second state information is a movement state and the first state information is a breathing state of the user, a weight may be given to a state in which the user is awake.

When both the first state information and the second state information indicate the presence of the user, but the movement of the user is not detected or breathing is detected, a weight may be assigned to the sleep state.

In an embodiment, the signal processing unit 720 may determine the state weights corresponding to the first state information and the second state information obtained from the current Wi-Fi signal and the acoustic signal, by using a pre-stored matrix in the form as shown in FIG. 9.

In an embodiment, the signal processing unit 720 may receive the first state information and the second state information for a predetermined time and determine a state weight corresponding thereto.

The signal processing unit 720 may analyze and classify the state weights accumulated for a predetermined time to extract time-series features of the state weights and obtain a final state therefrom.

In an embodiment, the signal processing unit 720 may use a neural network to obtain the final state. For example, the signal processing unit 720 may use a long short-term memory model (LSTM). The LSTM is a type of recurrent neural network (RNN) capable of learning long-term dependency between time steps of sequence data. The LSTM may receive sequence or time-series data and learn long-term dependency between time steps of the sequence data.

The signal processing unit 720 may input data accumulated in the neural network and obtain a final state therefrom. In an embodiment, the final state may be one of a user absence state, a user sleep state, and a user awake state.

In an embodiment, the signal processing unit 720 may obtain the final state of a user and transmit the final state to the mode changing unit 730.

Referring back to FIG. 7, when the signal processing unit 720 transmits the final state of a user to the mode changing unit 730, the mode changing unit 730 according to an embodiment may perform an operation of changing a mode based on the final state of a user. The mode changing unit 730 may be referred to as a state changing unit.

In an embodiment, the electronic device 100 may operate in the sleep mode or the normal mode depending on the operation of an element included in the electronic device 100.

The normal mode may refer to a state in which power is supplied to all components of the electronic device 100. In the normal mode, as each component of the electronic device 100 operates normally, the user may normally use the electronic device 100 based on a function, such as watching video content and audio content using the electronic device 100.

The sleep mode may be a low power mode and may include a standby mode.

In an embodiment, the sleep mode may be a mode in which power of the electronic device 100 is saved. In the sleep mode, power may be supplied only to components of the electronic device 100 that obtain and process a Wi-Fi signal or an acoustic signal, whereas power supply to other components may be cut off. As the network function operates even when the electronic device 100 is in the sleep mode, a communication unit may obtain a Wi-Fi signal. In addition, even in the sleep mode, the electronic device 100 may obtain and process an acoustic signal through the microphone array.

In an embodiment, the mode changing unit 730 may operate in the sleep mode or the normal mode based on the final state.

In an embodiment, the mode changing unit 730 may continue to operate in the normal mode when the final state of a user is the awake state.

In an embodiment, when the final state is the sleep state or the user absence state, the mode change unit 730 may change the mode from the normal mode to the sleep mode. In an embodiment, the mode changing unit 730 may control the display and the speaker not to output content by turning off the display and the speaker.

In an embodiment, while operating in the sleep mode switched from the normal mode in response to the final state of a user being the sleep state, the mode changing unit 730 may switch the mode back to the normal mode upon detecting that the user has woken up.

In an embodiment, while operating in the sleep mode switched from the normal mode in response to the user's sleep state, the mode changing unit 730 may transition the mode to a music mode or a meditation mode upon detecting that the user has woken up.

For example, the meditation mode or the music mode may be a mode for outputting a music signal of a low volume suitable for a user who has just woken up. The meditation mode may be a mode in which only audio content is output without video content, or may be a mode in which video content is output together with music.

However, this is merely an embodiment, and the mode changing unit 730 may operate in various modes corresponding to the final state.

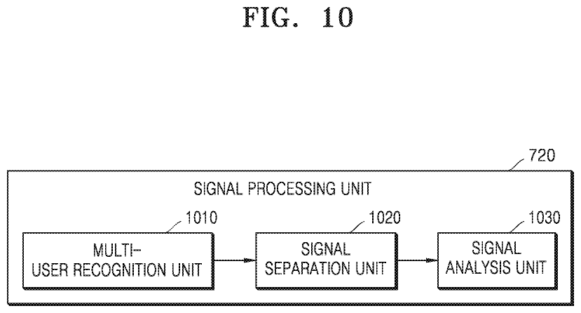

FIG. 10 is a block diagram according to an example of the signal processing unit 720 of FIG. 7.

Referring to FIG. 10, the signal processing unit 720 may include a multi-user recognition unit 1010, a signal separation unit 1020, and a signal analysis unit 1030, in the form of modules.

The multi-user recognition unit 1010 according to an embodiment may be a module for identifying whether there are a plurality of users.

In an embodiment, the multi-user recognition unit 1010 may detect that a plurality of users are present based on at least one of an acoustic signal of a Wi-Fi signal.

As described above, the Wi-Fi signal tends to increase in signal strength as the number of users increases.

In an embodiment, the multi-user recognition unit 1010 may detect a plurality of users based on at least one of the variation range or the signal strength of the Wi-Fi signal. In an embodiment, the multi-user recognition unit 1010 may identify the number of users by using the variation range of the Wi-Fi signal, that is, the strengths of a maximum value and a minimum value of the Wi-Fi signal.

In an embodiment, the multi-user recognition unit 1010 may identify that there are a plurality of users by using an acoustic signal. For example, the multi-user recognition unit 1010 may infer that there are a plurality of users based on at least one of a breathing event or an utterance/voice event of the plurality of users being detected in the acoustic signal.

In an embodiment, the multi-user recognition unit 1010 may infer the number of users from a CNN-based neural network previously trained on an acoustic signal in which one breathing sound or two or three breathing sounds are mixed.

In an embodiment, when voice activity detection (VAD) is performed on an acoustic signal, the multi-user recognition unit 1010 may segment the voice sections and extract speaker features for each segment.

In an embodiment, the multi-user recognition unit 1010 may adaptively train a universal background model (UBM) for each voice segment, and treat the adapted model as a distribution of the speaker's voice feature.

Alternatively, in an embodiment, the multi-user recognition unit 1010 may extract a speaker feature vector based on a deep learning model. The deep learning model used by the multi-user recognition unit 1010 may be trained by defining a distance-based objective function that induces the speaker vectors to be close to each other in the case of the same speaker and to be far from each other in the case of other speakers. The multi-user recognition unit 1010 may classify the speaker feature vectors based on distances.

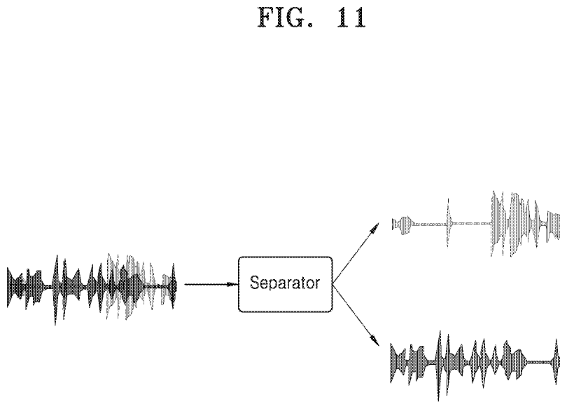

FIG. 11 is a diagram illustrating separating a voice signal into voice signals for each user, according to an embodiment.

The multi-user recognition unit 1010 of FIG. 10 may identify a user utterance section from an acoustic signal and extract a voice feature from the user utterance section. The multi-user recognition unit 1010 may identify that there are two users as illustrated in FIG. 11 by separating signals having similar characteristics.

Referring back to FIG. 10, when it is determined that there are a plurality of users based on the Wi-Fi signal and/or the acoustic signal, the multi-user recognition unit 1010 may inform the signal separation unit 1020 of the determination

The signal separation unit 1020 according to an embodiment may separate each of the acoustic signal and the Wi-Fi signal into a signal for each user when receiving information indicating that there are a plurality of users from the multi-user recognition unit 1010.

In an embodiment, the signal separation unit 1020 may collect a voice signal through a plurality of microphones. For example, when four microphones are arranged in a line in front of the electronic device 100 and two users are present around the electronic device 100, the signal separation unit 1020 may collect voice signals of the plurality of users by using the plurality of microphones.

In an embodiment, the signal separation unit 1020 may identify, for each microphone position, a user whose voice is received at a high signal level at each microphone. In the above example, the acoustic signal generated from the right user located on the right side of the microphone array is received at a high signal level in two right microphones among the four microphones and at an exponentially low signal level in the remaining two left microphones.

In an embodiment, the signal separation unit 1020 may separate, for each user, the acoustic signal corresponding to the user based on a structural characteristic of the microphone array and a pattern of the audio signal.

In an embodiment, the signal separation unit 1020 may group channels having similar characteristics based on a user's location and a feature based on the acoustic signal from the user.

In an embodiment, the signal separation unit 1020 may obtain an audio feature for each group by amplifying a frequency collected for each group and then filtering and correcting frequencies of other groups.

For example, in the above example, the signal separation unit 1020 may amplify channel signals obtained by two right microphones and including a large amount of acoustic signal features by a right side user and remove signals obtained by the remaining two left microphones from the amplified channel signals, thereby separating a first acoustic signal generated from a first user on the right from a second acoustic signal generated from a second user.

Similarly, out of the four microphones, the signal separation unit 1020 may separate the second acoustic signal originated from the second user on the left side from other acoustic signals, by amplifying channel signals obtained through the two microphones on the left and filtering the same using the signals obtained through the two microphones on the right.

In an embodiment, the signal separation unit 1020 may synchronize a Wi-Fi signal obtained simultaneously with sound information, that is, CSI raw data, with an audio signal.

In an embodiment, the signal separation unit 1020 may synchronize the acoustic signal and the CSI raw data by using an interval time between two signals indicating an input time difference between the acoustic signal and the Wi-Fi signal.

In an embodiment, the signal separation unit 1020 may identify a Wi-Fi signal having characteristics similar to those of an acoustic signal by using a neural network.

According to an embodiment, the signal separation unit 1020 may identify a first Wi-Fi channel corresponding to the first acoustic signal and identify a second Wi-Fi channel corresponding to the second acoustic signal.

In an embodiment, the signal separation unit 1020 may separate an acoustic signal and a Wi-Fi channel for each of a plurality of users, and may transmit the separated sound signal and Wi-Fi signal for each user to the signal analysis unit 1030.

In an embodiment, the signal analyzer 1030 may receive a signal separated for each user from the signal separation unit 1020 and obtain state information for each user based thereon.

In an embodiment, the signal analyzer 1030 may obtain first state information about the first user from the first acoustic signal and may obtain second state information about the first user from a first Wi-Fi channel corresponding to the first acoustic signal. In an embodiment, the signal analyzer 1030 may determine a state weight for the first user based on the first state information and the second state information.

Similarly, the signal analyzer 1030 may obtain the first state information about the second user from the second acoustic signal and obtain the second state information about the second user from the second Wi-Fi channel corresponding to the second acoustic signal. In an embodiment, the signal analyzer 1030 may determine a state weight for the second user based on the first state information and the second state information.

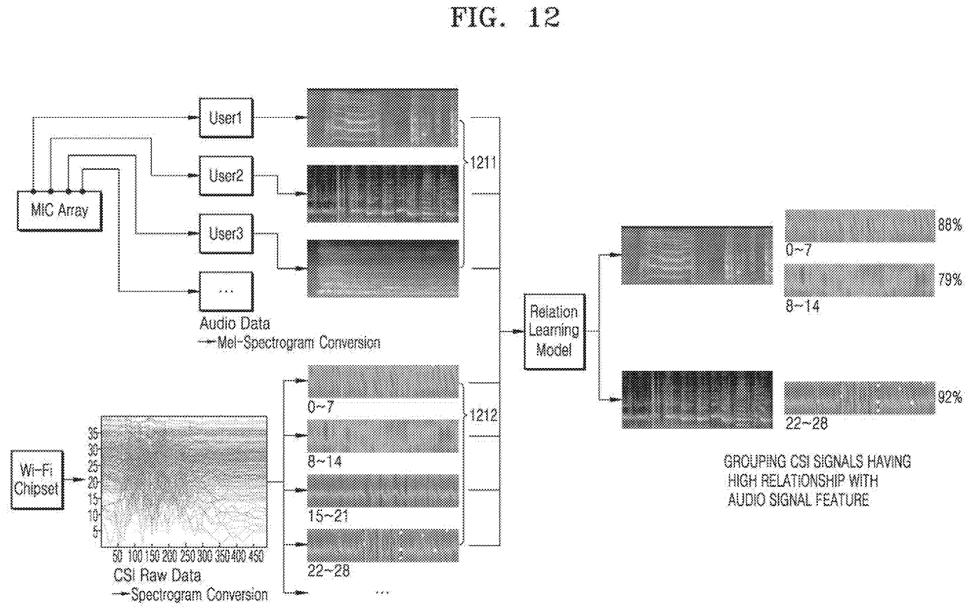

FIG. 12 is a diagram for describing that the signal separation unit 1020 of FIG. 10 identifies a Wi-Fi channel corresponding to an acoustic signal by using a neural network, according to an embodiment.

In an embodiment, the signal separation unit 1020 may use AI technology.

In order for a neural network, that is, a neural network, to accurately output result data corresponding to input data, it is necessary to train the neural network. Here, “training” may refer to training a neural network so that the neural network can discover or learn by itself a method of inputting various data into the neural network and analyzing the input data, a method of classifying the input data, and/or a method of extracting features necessary for generating result data from the input data.

Training a neural network means that an AI model of a desired characteristic is made by applying a training algorithm to a plurality of training data. In an embodiment, such training may be performed in the electronic device 100 itself in which AI is performed or may be performed through a separate server/system.

In an embodiment, the neural network used by the signal separation unit 1020 may be a neural network trained on the feature of an acoustic signal corresponding to each event, for each of various acoustic events, such as an utterance feature, a sound related to a movement, and a breathing sound. In addition, the neural network may be a neural network trained on a Wi-Fi signal pattern generated simultaneously with the corresponding acoustic signal, as training data, together with the acoustic signal feature.

In an embodiment, the neural network may be trained by using the acoustic signal pattern and the Wi-Fi signal pattern corresponding to the same event together, as training data.

For example, the neural network may be trained on a pattern of an acoustic signal when the user talks and a pattern of a Wi-Fi signal together, as one set, as training data. The neural network may be trained on a pattern of an acoustic signal when the user sits down and then stands up and a pattern of a Wi-Fi signal together, as one set, and may also be trained on an acoustic signal including a breathing sound when the user sleeps and a pattern of a Wi-Fi signal sensing a movement of the chest of the user at the same time point together, as one set.

In an embodiment, the neural network may obtain a feature of each of the acoustic signal pattern and the Wi-Fi signal pattern by using the acoustic signal pattern and the Wi-Fi signal pattern corresponding to the same event together, as training data, and may be trained on a relation between the obtained features, thereby being trained on a relation weight between the acoustic signal and the Wi-Fi signal.

In an embodiment, the neural network used by the signal separation unit 1020 may be a neural network trained to obtain Wi-Fi signals having pattern characteristics similar to those of acoustic signals from the acoustic signals.

In an embodiment, the signal separation unit 1020 may input a feature of a specific sound to the neural network that has been trained, and infer a Wi-Fi signal pattern highly related to the feature of the specific sound therefrom.

In an embodiment, the signal separation unit 1020 may identify a channel having a similar characteristics to a Wi-Fi signal pattern inferred using a neural network from among a plurality of Wi-Fi channels, and may select the identified channel or group the plurality of channels when there are a plurality of identified channels.

Hereinafter, an example in which the signal separation unit 1020 identifies a Wi-Fi channel corresponding to each user using a neural network will be described with reference to FIG. 12.

Referring to FIG. 12, the signal separation unit 1020 may separate an acoustic signal corresponding to a user, for each user, based on a structure characteristic of a microphone array and a pattern of an audio signal.

For example, as shown in FIG. 12, when the microphone array of the electronic device 100 includes four microphones, the signal separation unit 1020 may collect acoustic signals for each channel of the four microphones. The signal separation unit 1020 may collect an acoustic signal generated from the utterance or breathing characteristics of user 1 or the motion of user 1 from the acoustic signal collected by a first microphone among the four microphones. Similarly, the signal separation unit 1020 may collect acoustic signals of a second user (user 2) and a third user (user 3) through a second microphone and a third microphone, respectively.

In an embodiment, the signal separation unit 1020 may remove noise such as background noise and audio content output through the electronic device 100 from the acoustic signal for each user obtained by each microphone. The signal separation unit 1020 may convert the audio content and the acoustic signal for each user from which noise has been removed into Mel-spectrogram images 1211 and 1212 on the frequency domain.

The signal separation unit 1020 may obtain sound frequency features, such as pitch, timbre, and intensity, from the Mel-spectrogram image 1211 as an acoustic signal pattern.

In an embodiment, the signal separation unit 1020 may input a sound frequency feature for each user to the neural network.

In an embodiment, the signal separation unit 1020 may obtain a Wi-Fi signal synchronized with the acoustic signal. In an embodiment, the signal separation unit 1020 may receive a Wi-Fi signal synchronized with the acoustic signal, as CIS raw data, and preprocess the Wi-Fi signal to obtain an average value between a plurality of channels by grouping adjacent channels, except for a null channel. For example, the signal separation unit 1020 may group adjacent channels, such as channel groups 0 through 7, channel groups 8 through 14, channel groups 15 through 21, and channel groups 22 through 28.

In an embodiment, the signal separation unit 1020 may obtain a CSI amplitude value for each of the plurality of channels. The signal separation unit 1020 may generate the spectrogram image 1212 of a predetermined size by using the CSI amplitude value, and may process the spectrogram image into a data form that may be input to the neural network.

In an embodiment, the signal separation unit 1020 may input a Wi-Fi feature vector obtained from the spectrogram image 1212 to the neural network.

In an embodiment, the neural network that has been trained may obtain, as a result, a Wi-Fi signal pattern having a high relationship with the acoustic signal pattern for each user, based on a relationship between the acoustic signal pattern and the Wi-Fi signal pattern.

For example, as illustrated in FIG. 12, the neural network may identify channel groups 0 through 7 as Wi-Fi channels highly related to the acoustic signal of user 1, and identify channel groups 8 through 14 as Wi-Fi channels highly related to the acoustic signal of user 2.

In an embodiment, the signal separation unit 1020 may obtain first state information corresponding to user 1 from the acoustic signal of user 1, and may also obtain second state information corresponding to user 1 from the Wi-Fi signals included in channel groups 0 through 7.

Similarly, the signal separation unit 1020 may obtain second state information corresponding to user 2 from the acoustic signal of user 2, and may also obtain second state information corresponding to user 2 from the Wi-Fi signals included in channel groups 8 through 14.

As described above, according to an embodiment, the electronic device 100 may obtain a Wi-Fi signal corresponding to an acoustic signal of each of a plurality of users by using a neural network trained on a relationship between an acoustic signal pattern and a Wi-Fi signal pattern corresponding to the same event.

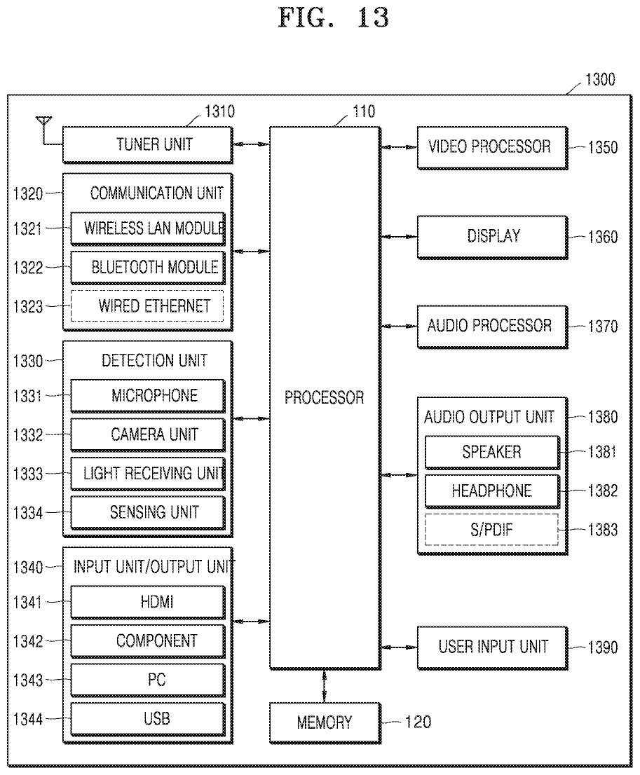

FIG. 13 is an internal block diagram of an electronic device according to an embodiment.

The electronic device 1300 of FIG. 13 may be an example of the electronic device 100 of FIG. 2. Hereinafter, redundant descriptions of those described with reference to FIG. 2 are omitted.

Referring to FIG. 13, the electronic device 1300 may further include a tuner unit 1310, a communication unit 1320, a detection unit 1330, an input unit/output unit 1340, a video processor 1350, a display 1360, an audio processor 1370, an audio output unit 1380, and a user input unit 1390, in addition to the processor 110 and the memory 120.

The tuner unit 1310 may tune and select only a frequency of a channel to be received by the electronic device 1300 from among many radio wave components through amplification, mixing, resonance, or the like of broadcast content or the like received in a wired or wireless manner. The content received through the tuner unit 1310 is decoded and separated into audio, video, and/or additional information. The separated audio, video, and/or additional information may be stored in the memory 120 under the control of the processor 110.

The communication unit 1320 may, under the control of the processor 110, connect the electronic device 1300 to a peripheral device, an external device, a server, a mobile terminal, or the like. The communication unit 1320 may include at least one communication module capable of performing wireless communication. The communication unit 1320 may include at least one of a wireless LAN module 1321, a Bluetooth module 1322, or a wired Ethernet 1323 based on the performance and structure of the electronic device 1300.

The wireless LAN module 1321 may transmit and receive a Wi-Fi signal to and from a peripheral device according to a Wi-Fi communication standard.

The Bluetooth module 1322 may receive a Bluetooth signal transmitted from a peripheral device according to a Bluetooth communication standard. The Bluetooth module 1322 may be a Bluetooth low energy (BLE) communication module and may receive a BLE signal. The Bluetooth module 1322 may continuously or temporarily scan a BLE signal to detect whether the BLE signal is received.

The detection unit 1330 may detect a user's voice, a user's image, or a user's interaction, and may include a microphone 1331, a camera unit 1332, a light receiving unit 1333, and a sensing unit 1334. The microphone 1331 may receive an audio signal including a voice uttered by a user or noise, convert the received audio signal into an electrical signal, and output the electrical signal to the processor 110.

The camera unit 1332 may include a sensor and a lens, capture an image formed on the screen, and transmit the captured image to the processor 110.

The light receiving unit 1333 may receive an optical signal (including a control signal). The light receiving unit 1333 may receive an optical signal corresponding to a user input (e.g., a touch, a press, a touch gesture, a voice, or a motion) from a control device such as a remote controller or a mobile phone.

The sensing unit 1334 may sense a state around the electronic device and transmit sensed information to the communication unit 1320 or the processor 110.

The input unit/output unit 1340 may, under the control of the processor 110, receive video (e.g., a moving image signal, a still image signal, or the like), audio (e.g., a voice signal, a music signal, or the like), additional information, or the like from a device or the like outside the electronic device 1300.

The input/output unit 1340 may include one of an HDMI port 1341, a component jack 1342, a PC port 1343, and a USB port 1344. The input unit/output unit 1340 may include a combination of the HDMI port 1341, the component jack 1342, the PC port 1343, and the USB port 1344.

The video processor 1350 may process image data to be displayed by the display 1360 and perform various image processing operations on the image data, such as decoding, rendering, scaling, noise filtering, frame rate conversion, and resolution conversion.

The display 1360 may output, on the screen, content received from a broadcasting station or received from an external server or an external storage medium. The content may include a video signal, an image, a text signal, or the like, as a media signal.

When the display 1360 is implemented as a touch screen, the display 1360 may be used as an input device such as a user interface, in addition to an output device. For example, the display 1360 may include at least one of a liquid crystal display (LCD), a thin film transistor-liquid crystal display (TFT-LCD), an organic light-emitting diode (OLED), a flexible display, a three-dimensional (3D) display, or an electrophoretic display. According to an implementation form of the display 1360, two or more displays 1360 may be included.

The audio processor 1370 processes audio data. The audio processor 1370 may perform various types of processing, such as decoding, amplification, or noise filtering, on the audio data.

The audio output unit 1380 may, under the control of the processor 110, output audio included in content received through the tuner unit 1310, audio input through the communication unit 1320 or the input unit/output unit 1340, or audio stored in the memory 120. The audio output unit 1380 may include at least one of a speaker 1381, a headphone 1382, or a Sony/Philips digital interface (S/PDIF) 1383.

In an embodiment, the audio output unit 1380 may output audio content while the electronic device 1300 operates in the normal mode.

The user input unit 1390 may receive a user input for controlling the electronic device 1300. The user input unit 1390 may include various types of user input devices including a touch panel for detecting a user's touch, a button for receiving a user's push operation, a wheel for receiving a user's rotation operation, a keyboard, a dome switch, a microphone for voice recognition, a motion detection sensor for sensing a motion, or the like, but is not limited thereto. When a remote controller or other mobile terminal controls the electronic device 1300, the user input unit 1390 may receive a control signal received from the mobile terminal.

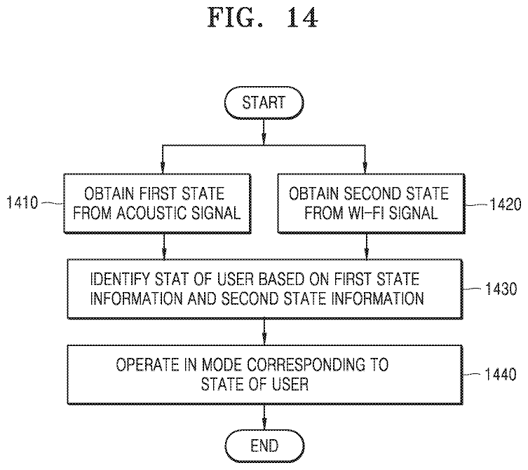

FIG. 14 is a flowchart illustrating an operating method of an electronic device, according to an embodiment.

Referring to FIG. 14, the electronic device 100 may obtain first state information from an acoustic signal (operation 1410).

In an embodiment, the electronic device 100 may filter audio content output by the electronic device 100 and other noise from the acoustic signal, and convert the filtered acoustic signal into a Mel-spectrogram image. The electronic device 100 may obtain a frequency feature of a sound from the Mel-spectrogram image, and obtain the first state information from the frequency feature of the sound.

In an embodiment, the electronic device 100 may obtain second state information from a Wi-Fi signal (operation 1420).

In an embodiment, the electronic device 100 may obtain a Wi-Fi signal pattern from the Wi-Fi signal and obtain the second state information by using the Wi-Fi signal pattern.

In an embodiment, the electronic device 100 may identify a state of a user based on the first state information and the second state information (operation 1430).

In an embodiment, the electronic device 100 may operate in a mode corresponding to the state of a user (operation 1440).

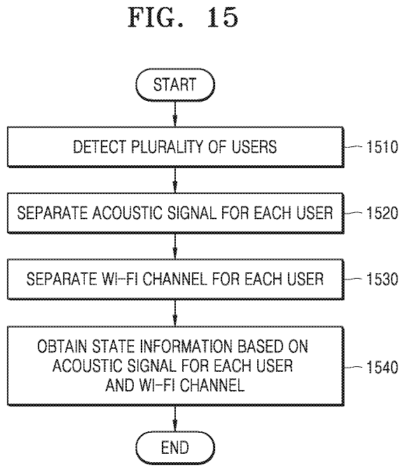

FIG. 15 is a flowchart illustrating an operating method of an electronic device, according to an embodiment.

Referring to FIG. 15, the electronic device 100 may include detecting a plurality of users (operation 1510)

In an embodiment, the electronic device 100 may detect a plurality of users based on at least one of an acoustic signal or a Wi-Fi signal.

In an embodiment, the electronic device 100 may separate an acoustic signal for each user in response to the detection of the plurality of users (operation 1520).

In an embodiment, the electronic device 100 may separate a Wi-Fi channel for each user (operation 1530).

In an embodiment, the electronic device 100 may identify a Wi-Fi channel corresponding to an acoustic signal of a certain user by using a neural network trained on a relationship between the acoustic signal and the Wi-Fi signal.

In an embodiment, the electronic device 100 may obtain state information based on the acoustic signal for each user and the Wi-Fi channel (operation 1540).

For example, the electronic device 100 may obtain first state information from an acoustic signal corresponding to the first user, obtain second state information from a Wi-Fi channel corresponding to the first user, and determine the final state of the first user by using the first state information and the second state information.

The operating method of an electronic device, and the electronic device, according to some embodiments, may be implemented in the form of a recording medium including instructions executable by a computer, such as a program module executed by a computer. A computer-readable storage medium may be a usable medium that is accessible by a computer and may include all of volatile and non-volatile media and separable and inseparable media. Furthermore, the computer-readable medium may include all of computer storage media and communication media. The computer storage media may include all of volatile and non-volatile media and separable and inseparable media, which are embodied by a certain method or technology for storing information such as computer-readable instructions, data structures, program modules, or other data. The communication media may typically include computer-readable instructions, data structures, program modules, or other data of a modulated data signal such as a carrier wave, or other transmission mechanism, and may also include information transmission media.

In addition, the electronic device and the operating method thereof according to an embodiment of the present disclosure described above may be implemented as a computer program product including a computer-readable recording medium/storage medium having recorded thereon a program for implementing an operating method of an electronic device, the operating method including: obtaining first state information using an acoustic signal pattern obtained from an acoustic signal; obtaining second state information (presence or absence of a user, a motion of a user) using a Wi-Fi signal pattern obtained from a Wi-Fi signal; identifying a state of the user based on the first state information and the second state information; and operating in a mode corresponding to the state of the user.

The machine-readable storage medium may be provided in the form of a non-transitory storage medium. Here, the non-transitory may mean that a storage medium is a tangible device, not including a signal (e.g., an electromagnetic wave). However, the term does not distinguish a case of semi-permanently storing data in a storage medium from a case of temporarily storing data. For example, the “non-transitory storage medium” may include a buffer in which data is temporarily stored.

According to an embodiment, a method according to various embodiments disclosed herein may be included and provided in a computer program product. The computer program product may be traded between a seller and a buyer as a commodity. The computer program product may be distributed in the form of a machine-readable storage medium (e.g., a compact disc read only memory (CD-ROM)), or may be distributed (e.g., downloaded or uploaded) online through an application store or directly between two user devices (e.g., smartphones). In the case of online distribution, at least a portion of a computer program product (e.g., a downloadable app) may be at least temporarily stored in a machine-readable storage medium such as a memory of a manufacturer's server, a server of an application store, or a relay server, or may be temporarily generated.

What is claimed is:

1. An electronic device comprising:
memory storing at least one instruction; and
at least one processor,
wherein the at least one instruction, when executed by the at least one processor individually or collectively, causes the electronic device to:
identify a final state of at least one user based on first state information and second state information, the first state information being obtained based on an acoustic signal pattern obtained from an acoustic signal, and the second state information being obtained based on a Wi-Fi signal pattern obtained from a Wi-Fi signal; and
control the electronic device to operate in a state corresponding to the final state of the at least one user.

2. The electronic device of claim 1, wherein the at least one instruction, when executed by the at least one processor individually or collectively, causes the electronic device to, based on a distance to a generation point of the acoustic signal being within a preset range, operate in the state corresponding to the final state of the at least one user.

3. The electronic device of claim 1, further comprising a speaker,
wherein the at least one instruction, when executed by the at least one processor individually or collectively, causes the electronic device to:
obtain a Mel-spectrogram image based on the acoustic signal, from which noise is filtered, and audio content, output through the speaker; and
obtain the first state information based on a frequency feature of a sound, the frequency feature of the sound being obtained based on the Mel-spectrogram image, and
wherein the frequency feature of the sound comprises at least one of a pitch, a timbre, or an intensity of the sound.

4. The electronic device of claim 1, wherein the at least one instruction, when executed by the at least one processor individually or collectively, causes the electronic device to:
obtain the Wi-Fi signal pattern from the Wi-Fi signal; and
obtain the second state information based on a pattern trend of the Wi-Fi signal obtained for a predetermined time.

5. The electronic device of claim 1, wherein the at least one instruction, when executed by the at least one processor individually or collectively, causes the electronic device to,
based on a plurality of users being identified based on at least one of the acoustic signal or the Wi-Fi signal, identify a corresponding signal for each of the plurality of users based on the acoustic signal and the Wi-Fi signal.

6. The electronic device of claim 5, wherein the at least one instruction, when executed by the at least one processor individually or collectively, causes the electronic device to perform at least one of: identifying the plurality of users based on at least one of a variation range or a signal strength of the Wi-Fi signal; and
identifying the plurality of users based on at least one of voice events of the plurality of users or breathing events of the plurality of users in the acoustic signal.

7. The electronic device of claim 5, wherein the at least one instruction, when executed by the at least one processor individually or collectively, causes the electronic device to:
obtain the acoustic signal based on a plurality of microphones;
obtain, based on the acoustic signal, a first acoustic signal including an utterance feature of a first user; and
obtain the first state information corresponding to the first user based on the first acoustic signal.

8. The electronic device of claim 7, wherein the at least one instruction, when executed by the at least one processor individually or collectively, causes the electronic device to:
identify a first Wi-Fi channel corresponding to the first acoustic signal based on a neural network trained on a relationship between acoustic signals and Wi-Fi signals; and
obtain the second state information corresponding to the first user from the first Wi-Fi channel.

9. The electronic device of claim 8, wherein the at least one instruction, when executed by the at least one processor individually or collectively, causes the electronic device to:
identify a state weight of the first user based on the first state information and the second state information corresponding to the first user; and
identify a final state of the first user based on the state weight of the first user obtained for a predetermined time.

10. The electronic device of claim 1, wherein the at least one instruction, when executed by the at least one processor individually or collectively, causes the electronic device to:
control the electronic device to operate in a device sleep state based on identifying that the final state for each of the at least one user is a user sleep state.

11. An operating method of an electronic device, the operating method comprising:
identifying a final state of at least one user based on first state information and second state information, the first state information being obtained based on an acoustic signal pattern obtained from an acoustic signal, and the second state information being obtained based on a Wi-Fi signal pattern obtained from a Wi-Fi signal; and
operating the electronic device in a state corresponding to the final state of the at least one user.

12. The operating method of claim 11, wherein the operating the electronic device comprises, based on a distance to a generation point of the acoustic signal being within a preset range, operating the device in the state corresponding to the final state of the at least one user.

13. The operating method of claim 11, further comprising outputting audio content,
wherein the obtaining the first state information comprises:
obtaining a Mel-spectrogram image based on the acoustic signal, from which noise is filtered, and the audio content; and
obtaining the first state information based on a frequency feature of a sound, the frequency feature of the sound being obtained based on the Mel-spectrogram image, and
wherein the frequency feature of the sound comprises at least one of a pitch, a timbre, or an intensity of the sound.

14. The operating method of claim 11, wherein the obtaining of the second state information comprises:
obtaining the Wi-Fi signal pattern from the Wi-Fi signal; and
obtaining the second state information based on a pattern trend, of the Wi-Fi signal, obtained for a predetermined time.

15. A non-transitory computer-readable recording medium having recorded thereon a program that is executed by at least one processor to perform the operating method of claim 11.
